(19) United States
(12) Patent Application Publication
(10) Pub. No.: US 2026/0273175 A1
(43) Pub. Date: Sep. 17, 2026

(54) Programmable Device for Precise Medication Delivery

(71) Applicant: BIO HEALTH FRONTIERS, INC., El Dorado Hills, CA (US)

(72) Inventors: Paul Lambert, El Dorado Hills, CA (US); Carlos Gutierrez, El Dorado Hills, CA (US); Travis Schraeder, El Dorado Hills, CA (US); Neal de Beer, El Dorado Hills, CA (US); JonPaul Lambert, El Dorado Hills, CA (US); Kenneth Tallo, El Dorado Hills, CA (US); Samuel Miller, El Dorado Hills, CA (US)

(21) Appl. No.: 19/082,007

(22) Filed: Mar. 17, 2025

(51) Int. Cl. A61M 5/145; A61M 5/142

(52) U.S. Cl. CPC A61M 5/145; A61M 2005/14208

(57) ABSTRACT

An infusion device includes a medication container interface to couple with a first portion of a medication container. The medication container has a second portion having an orifice and a medication chamber that encapsulates fluid which is disposed between the first and second portions of the medication container. A pressure chamber is formed when the first portion of the medication container is interfaced with the medication container interface. A pressure source, when active, expels gas and/or fluid within the pressure chamber to apply a pressure to a surface of the medication container causing fluid within the medication chamber to be expelled out of the orifice. A pressure sensor measure pressures within the pressure chamber. A control unit in communication with the pressure source and the pressure sensor and sends signals to the pressure source to control infusion parameters based, at least in part on, the measured pressure.

### TECHNICAL FIELD

The subject matter described herein relates to compact, hand-held, and programmable devices for precisely infusing medications such as immunoglobulin from medication containers such as syringes.

### BACKGROUND

Precise control of drug delivery is critical for patient safety and optimizing treatment efficacy. A vast array of infusion devices has been developed including hospital based, home based and ambulatory devices.

In the ambulatory category, prevalent types of utilized volumetric pumps include peristaltic pumps, lead screw-type syringe drivers and mechanical pumps. The first two technologies are electromechanical pumps engineered to deliver a controlled volume of fluid over a designated period, thereby ensuring accurate flow rates for drug administration. In contrast, although mechanical pumps can infuse at relatively constant flow rates, have important limitations and present issues when precise flow control is required.

Cost per infusion is a significant factor that determines what pump technology and pump design is selected for a specific therapy. The spectrum of cost is wide and user cases are not always able to benefit from using a specific pump that meets budget limitations.

Peristaltic pumps are widely used in medical applications due to their ability to accurately control fluid delivery without direct contact between the fluid and the pump mechanism. These pumps operate by compressing and releasing a flexible tube with a rotating series of rollers or cams. As the rollers move along the tube, they create a peristaltic motion that propels the fluid forward. The flow rate is directly proportional to the speed of the rollers, allowing precise control of the volume delivered over time. This mechanism makes peristaltic pumps useful for situations in which contamination must be avoided, as the fluid only contacts the inner surface of the tubing.

Lead screw-type syringe drivers are another technology for controlled drug delivery. In these devices, a motor-driven lead screw advances or retracts the plunger of a syringe at a controlled rate. This controlled movement allows for exact dosing by delivering a specific volume of fluid over time, directly controlling the flow rate. Lead screw-type syringe drivers are useful in applications requiring high precision, such as in infusion therapy or patient-controlled analgesia.

With a lead screw-type syringe driver, the flow rate is controlled, however, the pump does not have the ability to adjust to dynamic biological variations. For example, if an infusion targets the subcutaneous tissue, during a flow rate-controlled infusion, the pump may produce a higher flow rate than the subcutaneous tissue can absorb. Maintaining a flow rate that is too high may lead to unsafe high-pressure conditions if tissue does not absorb the drug which is why flow rate-controlled pumps have occlusion alarms.

While peristaltic pumps and lead screw-type syringe drivers offer precise control over the volume of fluid delivered over time, resulting in accurate flow rates, constant force syringe pumps operate on a fundamentally different principle. These devices rely on a mechanical spring or other force-generating mechanism to apply a continuous, steady force to the syringe plunger, pushing fluid out at a rate determined by the system's resistance rather than by an actively controlled mechanism.

However, most mechanical constant force pumps (springs or elastomers) are subject to non-linearities and the force of a given pump convert into varying pressure levels depending on the geometry (different barrel diameters) of the syringes containing the medicine.

In volumetric pumps like peristaltic pumps and lead screw-type syringe drivers, the flow rate is directly controlled by the device. For peristaltic pumps, the flow rate is managed by the speed of the rollers, while in lead screw syringe drivers, it is determined by the motor's precise control over the lead screw's rotation. In both cases, the device ensures that a specific volume of fluid is delivered over a set period, with a high level of accuracy.

In contrast, a constant force syringe pump does not actively control the flow rate. The flow rate in these devices is a passive outcome of the constant force applied by the spring and the resistance encountered by the fluid as it travels through the delivery system, such as the tubing and needle. Factors like the viscosity of the fluid, the diameter of the tubing, and the needle gauge all influence the actual flow rate. As a result, while these devices can offer a steady delivery, they do not provide the precise volumetric control seen in peristaltic and lead screw-type pumps. With constant force pumps, the maximum upper limit to flow rate that will occur is limited by the force applied and the fluid path resistance, then any additional backpressure or resistance from the patient will slow the flow rate down from the maximum limit.

### SUMMARY

In a first aspect, an infusion device includes a housing encapsulating a pressure source, a pressure sensor, and a control unit. The housing defines a medication container interface having a shape and size to interface (i.e., secure, communicate, etc.) with a first portion of a medication container. The medication container has a second portion having an orifice and a medication chamber which encapsulates fluid and is disposed between the first and second portions of the medication container. A pressure chamber is formed when the first portion of the medication container is interfaced (i.e., coupled, etc.) with the medication container interface. The pressure source, when active, expels gas within the pressure chamber to apply a pressure to a surface of the medication container causing fluid within the medication chamber to be expelled out of the orifice. The pressure sensor is configured or otherwise positioned so as to measure pressure within the pressure chamber. The control unit is in communication with the pressure source and the pressure sensor and sends signals to the pressure source to control infusion parameters based, at least in part on, the measured pressure.

The medication container can take varying forms. In some variations, the first portion of the medication container comprises a movable surface or component. In other variations, the first portion of the medication container comprises a compressible or deformable surface. The second portion of the medication container can take different forms including being rigid, deformable, moveable and/or be a membrane.

The pressure source can take varying forms. For example, the pressure can be a pneumatic pump. In some variations, the pneumatic pump is a piezoelectric pump.

The medication container can be directly coupled to the medication container interface. In other variations, the medication container can be indirectly coupled to the medication container interface for example, by way of an adapter or tubing.

The medication container can have a flange on its first side and there can further be an adapter having one or more locking tabs positioned to allow insertion and securing of the first side of the medication container into the adapter. The adapter can have a shape and size to be received in and secured by the medication container interface.

The pressure generated by the pressure source can be directly applied to the surface of the medication container. The pressure generated by the pressure source can be indirectly applied to the surface of the medication container by way of one or more intermediate elements between the housing at the medication container.

The medication container can be a syringe having a plunger and the surface to which pressure is directly or indirectly applied is the plunger.

The medication container can also be a plunger-less syringe in which the pressure chamber is formed in the opening defined by the bottom portion and the stopper element. The plunger-less syringe can include at least one flange on the first portion and the medication container interface can have one or more locking tabs positioned to allow insertion and securing of the first portion of the medication container into the medication container interface.

The medication container can be secured by the one or more locking tabs after rotating from a first position to a second position.

The medication container can be an on-body injector adjustable to a contour of a body of a user. The medication container can be an injector pen. The medication can be a reservoir such as a flat reservoir. The medication container can be a bag.

The control unit can include a microprocessor and memory and can interface with the various components forming part of the infusion device in order to monitor, assess, and control infusion. The control unit can, for example, control infusion parameters using a locally stored look up table which correlates factors such as measured pressure, a geometry of the medication container, a viscosity of fluid (i.e., medication) contained within the medication container, and an infusion tubing set in communication with the orifice (i.e., the downstream infusion tubing and/or downstream needle set, etc.) with a predetermined flow rate.

The housing can also encapsulate a data store (e.g., memory, a memory device, etc.) to store infusion parameter measurements generated by the control unit. The infusion device and/or the control unit can include a central processing unit (CPU) which an be used to generate time measurements (e.g., time stamps, etc.) associated with the various pressure sensor measurements.

The infusion device can include a communications interface encapsulated within the housing and in communication with the data stored and the control unit. The communications interface can transmit data stored in the memory characterizing the infusion parameter measurements to a remote computing device. The communications interface can provided a wired and/or wireless communications to external computing devices (including network-connected medical devices and equipment, mobile phones, tablets, patient monitors, hospital data systems, computers, etc.). In some variations, the communications interface is bidirectional and receives signals from one or more external network-connected medical devices associated with a patient (i.e., the person receiving the fluid expelled from the orifice of the medication container, etc.). The remote computing device can send signals, by way of the communications interface, to cause the control unit initiate and stop expulsion of the fluid from the medication container.

The transmitted data can include fluid flow rate information or values to calculate the fluid flow rate information. The fluid flow rate information can be one or more of: time of infusion, fluid flow rate, fluid pressure profile, mode of operation by the pressure source, and patient compliance statistics.

The control unit can estimate and correlate detected pressure with one or more of volume infused, flow rate, and therapy settings relative to a type of the fluid and/or an infusion protocol (e.g., a programmed infusion protocol, etc.).

The control unit can be configured to send signals to the pressure source to change a pressure limit from a first value to a second value.

The control unit can be configured to send signals to the pressure source to cause the fluid to be expelled according to a first flow rate profile as well as to change a fluid pressure profile at which the fluid is expelled from the first pressure profile to a second, different flow rate profile.

The control unit, based on the measured pressure, and fluid characterization data, can determine (i) a condition at which all of the fluid in the medication container has been expelled through the orifice based on a level of pressure detected in the pressure chamber, (ii) an occlusion condition within an infusion set coupled to the medication container based on a detected build up of pressure within the pressure chamber, and/or (iii) that the medication container has been removed from the medication container interface based on a detected release of pressure within the pressure chamber.

The infusion device can further include a proximity sensor in or adjacent to the medication container interface which is configured to detect presence or absence of a medication container within the medication container interface and transmit corresponding signals to the control unit.

The infusion device can further include a position sensor in or adjacent to the medication container interface which is configured to detect changes correlated to a volume of the medication chamber and transmit corresponding signals to the control unit.

The infusion device can include a pressure relief valve encapsulated within the housing and in communication with the control unit which, when activated in response to a signal from the control unit, causes the pressure chamber to vent resulting in a cessation of fluid out of the orifice. The pressure relieve valve can take varying forms including a solenoid valve.

The infusion device can include a mechanical pressure relief valve encapsulated within the housing which causes the expulsion of the fluid out of the orifice to cease when a predetermined physical parameter condition (e.g., pressure within the pressure chamber exceeds a threshold, etc.) is met.

The infusion device can include an expandable element (e.g., inflatable O-ring, etc.) disposed within (e.g., circumferentially around the mount, etc.) or adjacent to the medication container interface. The pressure source or a second, different pressure source within the housing can cause the expandable element to inflate to secure the medication container within the medication container interface.

In some variations, the infusion device includes an expandable element disposed within or adjacent to the medication container interface which, rather than being used to sure the medication container, can be inflated to exert a mechanical force on the first side of the medication container when placed within the medication container interface. This expandable element can be inflated by the pressure source or a second, different pressure source within the housing.

The medication container interface can take different forms. In one variation, the medication includes slots having a shape and size complementary to flanges on the medication container which allow for axial rotation of the medication container. The medication container interface can include snaps to prevent the medication container from rotating once secured in the medication container interface. The medication container interface can have a series of stacked circumferential grooves having depths corresponding to differently sized medication container interfaces. In some cases, at least a portion of the medication container interface is integral to the housing. In other variations, the medication container interface is an external component secured to the housing.

The infusion device can include an air port extending through an external surface of the housing having a fitting for communicating with an external tubing set or device for receiving a source of gas. The source of gas can be the pressure source or a second, different pressure source within the housing.

In some variations, the infusion device can also include a reference chamber, a first temperature sensor, and a second temperature sensor. The reference chamber has a known, fixed volume. The first temperature sensor can be within the housing and in communication with the control unit to measure temperature within the pressure chamber. The second temperature sensor can be within the housing and in communication with the control unit to measure temperature within the reference chamber. The infusion parameters, in such case, are further controlled based on values generated by the first temperature sensor and the second temperature sensor.

In an interrelated aspect, the infusion device includes a housing encapsulating a pressure source and a control unit (and optionally omitting a pressure sensor). The housing defines a medication container interface having a shape and size to receive and secure a first portion of a medication container. The medication container includes an orifice on a second portion of the medication container. A pressure chamber is formed when the first portion of the medication container is secured to the medication container interface. The pressure, when active, expels gas within the pressure chamber to apply a pressure to a surface of the medication container causing fluid within the medication container to be expelled out of the orifice. The control is unit is in communication with the pressure source and sends signals to the pressure source to control infusion parameters.

In a further interrelated aspect, an infusion device includes a housing encapsulating a pressure source and a control unit and also include a pressure sensor external to the housing. The housing can define a medication container interface having a shape and size to receive and secure a first portion of a medication container. The medication container further comprises an orifice on a second portion of the medication container. A pressure chamber is formed when the first portion of the medication container is secured to the medication container interface. The pressure source, when active, expels gas within the pressure chamber to apply a pressure to a surface of the medication container causing fluid within the medication container to be expelled out of the orifice. The pressure sensor measures pressure of the fluid downstream after being expelled from the orifice. The control unit is in communication with the pressure source and the pressure sensor which, based on the measured pressure, sends signals to the pressure source to control infusion parameters.

In yet a further interrelated aspect, an infusion device includes a housing encapsulating a pressure source and a control unit. In addition, a flow sensor measures fluid flow from the medication container. The housing defines a medication container interface having a shape and size to receive and secure a first portion of a medication container. The medication container further comprises an orifice on a second portion of the medication container. A pressure chamber is formed when the first portion of the medication container is secured to the medication container interface. The pressure source when active, expels gas within the pressure chamber to apply a pressure to a surface of the medication container causing fluid within the medication container to be expelled out of the orifice. The flow sensor, which can be internal to the housing or external to the housing. With the latter, the flow sensor can measure fluid flow downstream from the orifice of the medication container. The control unit is in communication with the pressure source and the flow sensor and, based on the measured fluid flow, sends signals to the pressure source to control infusion parameters.

In still another interrelated aspect, an infusion device includes a housing encapsulating a pressure source, a reference chamber having a known volume, a first pressure sensor, a second pressure sensor, a first temperature sensor, and a second temperature sensor. The housing defines a medication container interface having a shape and size to receive and secure a first portion of a medication container. The medication container further comprises an orifice on a second portion of the medication container. A pressure chamber is formed when the first portion of the medication container is secured to the medication container interface. The pressure source, when active, expels gas within the pressure chamber to apply a pressure to a surface of the medication container causing fluid within the medication container to be expelled out of the orifice. The first pressure sensor measures pressure within the pressure chamber to generate a first pressure value. The second pressure sensor measures pressure within the reference chamber to generate a second pressure value. The first temperature sensor measures temperature within the pressure chamber to generate a first temperature value. The second temperature sensor measures temperature within the reference chamber to generate a second temperature value. The control unit is in communication with the pressure source, the first pressure sensor, the second pressure sensor, the first temperature sensor, the second temperature sensor. The control unit sends signals to the pressure source to control infusion parameters based on the first pressure value, the second pressure value, the first temperature value, and the second temperature value.

The subject matter described herein provides many technical advantages. For example, the current subject matter provides a hand-held infusion pump that allows a user to safely self-administer medication according to a prescribed regimen. Further, when tissue absorption rate is reduced, constant pressure force pumps will dynamically change their flow rate (up or down) based on a number of factors external to the pump and not the inherent design of the pump. The factors that affect the performance of mechanical pumps include the patient absorption rate design and geometry of the pump's accesories, and the variable drug viscosity—(a factor of temperature). On the other hand, pressure-based pumps have a similar dynamic effect on the flow rate. However, in pressure controlled pumps and with the current subject matter, there can be optimization of flow controlled by the pump through dynamic pressure adjustments as needed to optimize the infusion considering factors such as pump, accessories, patient and drug viscosity

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a diagram illustrating an infusion pump coupled to a medication container which, in turn, is connected to tubing and a needle set;

FIG. 2 is a diagram illustrating the infusion pump of FIG. 1 separated from the medication container, tubing, and needle set;

FIG. 3 is a diagram illustrating the infusion pump of FIG. 1;

FIG. 4 is a diagram illustrating an exploded view of the infusion pump of FIG. 1;

FIG. 5 is a first logical diagram illustrating components which can form part of the infusion pump of FIG. 1;

FIG. 6 is a second logical diagram illustrating components which can form part of the infusion pump of FIG. 1; and

FIGS. 7-10 are diagrams illustrating a medication container adapter allowing differing medication container sizes to be secured to the infusion pump of FIG. 1.

Like reference symbols in the various drawings indicate like elements.

### DETAILED DESCRIPTION

The current subject matter is directed to a compact, hand-held infusion pump that can precisely infuse fluid (e.g., medication, etc.) from medication containers such as syringes, medication containers having a deformable portion as well as rigid medication containers such as vials. Such a pump is particularly helpful for subcutaneous immunoglobulin (SCIg) treatments and the like which require sub-cutaneous infusion of medication at controlled flow rates.

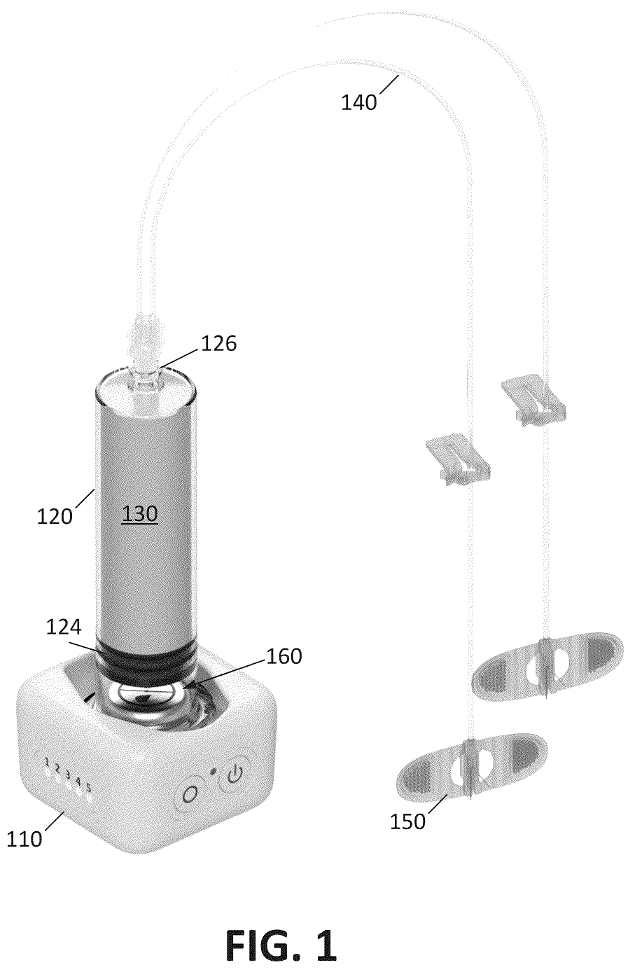

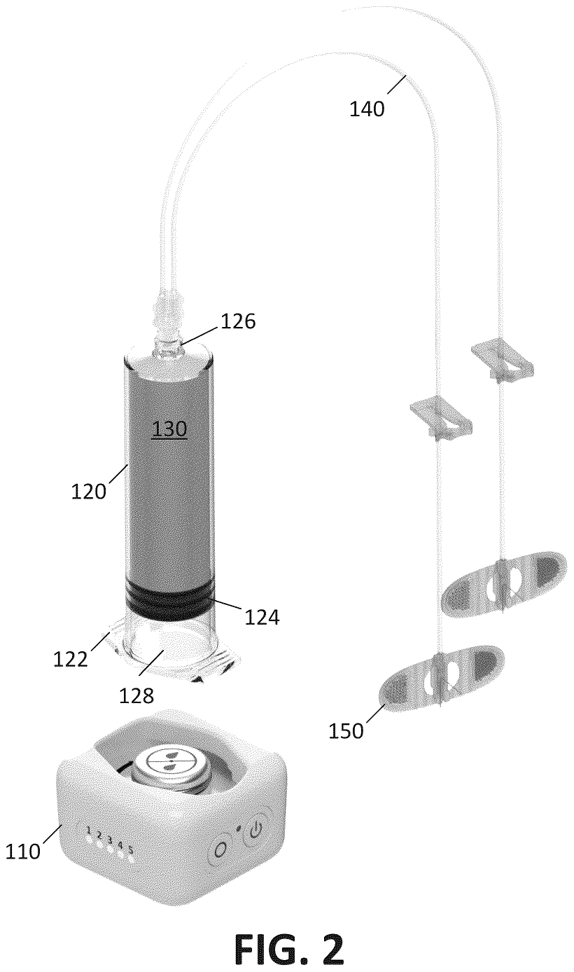

FIG. 1 is a diagram illustrating an arrangement in which an infusion pump 110 can receive and secure a medication container 120 and cause fluid (i.e., medication, etc.) contained therein to be infused to a patient by way of a tubing set 140 connected to or integrated with a needle set 150 (e.g., an intravenous needle set or a sub-cutaneous needle set, etc.). FIG. 2 is a diagram providing a view of the medication container 120 detached from the infusion pump 110. The medication container 120, in this variation, is a plunger-less syringe having one or more flanges 122, a rubber stopper 124, a medication chamber 130 (i.e., that portion of the medication container 120 in which fluid is housed), and an orifice 126. In some cases, the medication container 120 is a plunger-less syringe without flanges. Fluid contained within the medication chamber 130 is expelled when the rubber stopper 124 is translated in response to pressure (e.g., gas, etc.) generated by the infusion pump 110. An interior portion of the medication container 120 can be cylindrical/barrel shaped and include an opening 128 at an end opposite the orifice 126.

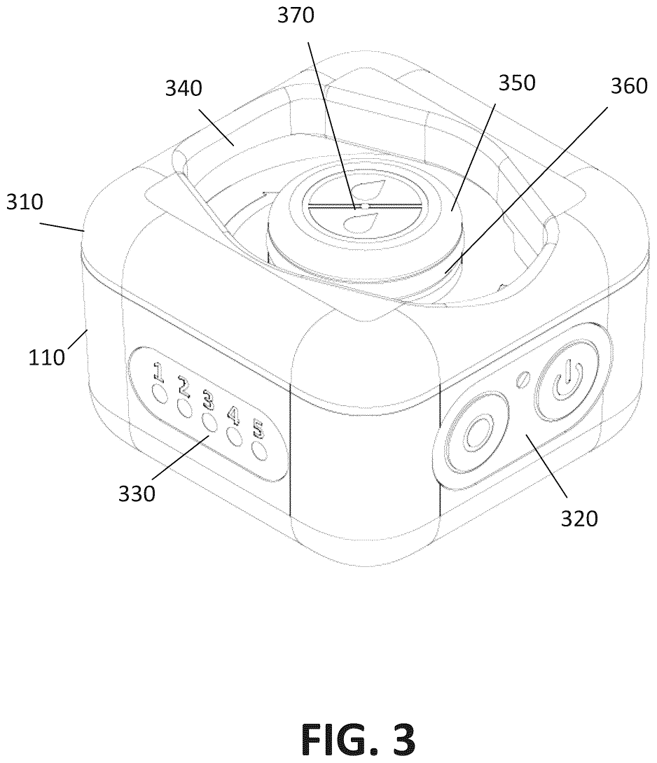

FIG. 3 is a diagram illustrating further details of the infusion pump 110. The infusion pump 110 can have a housing 310 with a shape and size to allow it to be handheld. In some variations, the housing 310 has a length and width of 8 cm or less and a height of 5 cm or less. The size of the housing can also vary based on the size of the utilized medication container (e.g., plunger-less syringe) and in some cases the housing 310 can be larger or in other cases it can be smaller. The external face of the housing 310 can include various manually-activated user control elements including power on/off buttons 320 and varying infusion setting elements 330. The infusion setting elements 330 can, for example, correspond to different infusion lengths (e.g., 1 minute, 5 minutes, 30 minutes, etc.) medications, prescription information, compatible supplies, and/or other infusion parameters (e.g., flow rate profiles, etc.). The infusion settings corresponding to the setting elements 330 can be programmable or otherwise modifiable by a user. For example, the infusion parameters for the settings elements 330 can be programmed by way of a remote computing device in communication with the infusion pump 110 (e.g., a mobile phone, tablet, remote computer, or other).

The housing 310 can form a medication container interface 340 which has a shape and size to receive and secure the medication container 120. The infusion pump 110 can additionally include a mount 350 (i.e., a medication container mount) having a shape and size to receive the opening 128 of the medication container 120. In the cases of the medication container 120 being a syringe or other receptacle with flanges 122, the medication container 120 can be positioned so that the mount 350 is inserted into the opening 128. The medication container 120 can then be rotated (e.g., clockwise) so that the flanges 122 contact or are otherwise secured by one or more securing elements forming part of the medication container interface 340. The securing elements can take varying forms including: locking tabs complementary to, for example, the flanges 122 and/or snaps to prevent the medication container 120 from rotating once it is secured. In some variations, the medication container interface 340 can include slots having a shape and size complementary to flanges 122 on the medication container 120 to allow for insertion and removal of the medication container 120 when not secured. In some variations, the medication container interface 310 can include a series of stacked circumferential grooves having depths corresponding to differently sized medication containers 120 (e.g., different syringe sizes, different flange sizes and positions, etc.).

The mount 350 can include one or more seals 360 (e.g., gaskets, O-rings etc.) on an outer circumference/surface to create an air-tight seal with the opening 128 of the medication container 120. The medication container 120 when secured to the medication container interface 340 forms a pressure chamber 160 in the open space within the opening 128 between the mount 350 and the plunger 124. In some variations, the one or more seals 360 are expandable so as to fit different sizes and/or tolerances of medication containers 120. For example, a pressure source 505 can cause the seal 360 to be inflated and thus expand to create the pressure chamber 160 within the medication container 120. In other variations, a second pressure source can be used to inflate the seal 360. The mount 350 can also include a valving mechanism 370 in which gas can be passed therethrough (as described in more detail below) into the pressure chamber.

In some variations, the mount 350 can have stacked and stepped ridges each having a decreasing diameter (relative to the distal portion of the mount 350) each having a corresponding seal 360. Such an arrangement can be used with differently sized medication containers 120 optionally without the need for any sort of adapter (such as those described below).

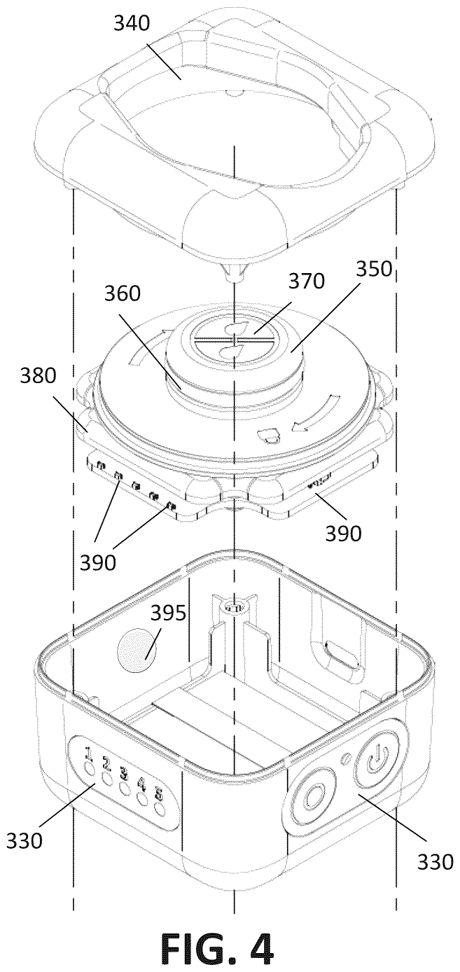

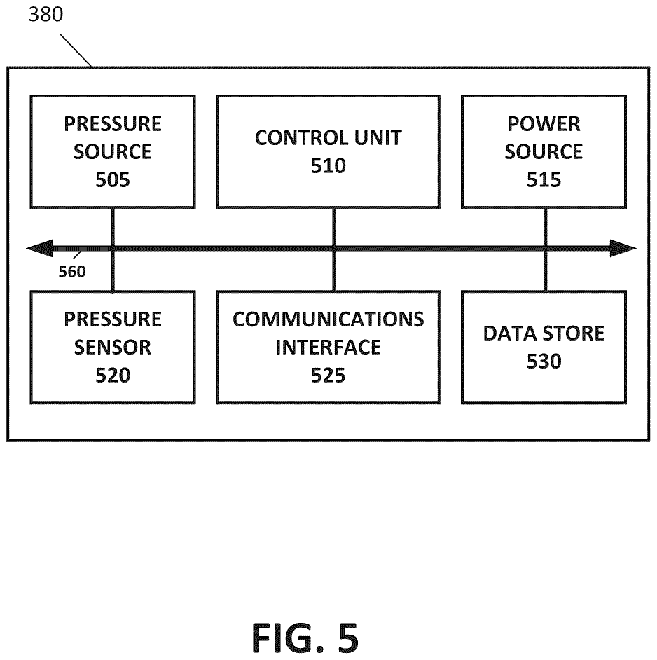

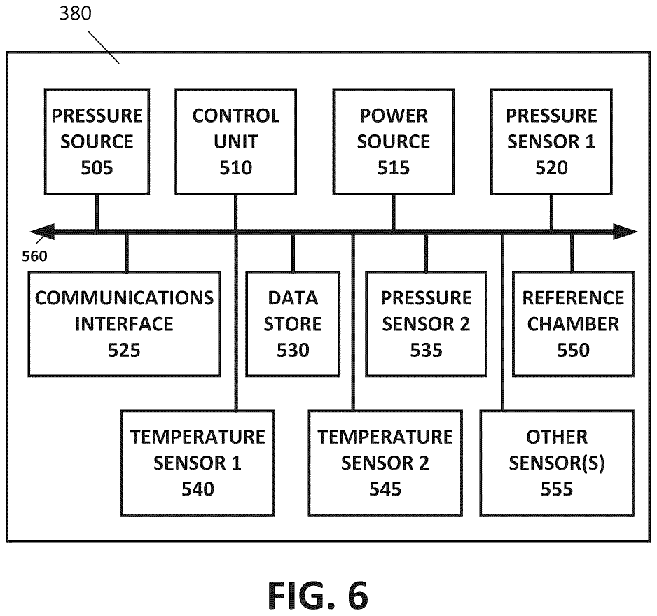

FIG. 4 is an exploded view of the infusion pump 110 which illustrates a component platform 380 on which various components 505-555 (illustrated logically in FIGS. 5 and 6) can be mounted and electrically connected, etc. In some cases, the various components 505-555 can be connected by way of a bus 560. Other configurations with direct communications amongst the components 505-555 can also be implemented. The component platform 380 allows for all of the components 505-555 to be encapsulated within the housing 310. Different mounting and connectivity arrangements can also be utilized provided that the components 505-555 are encapsulated within the housing 310.

The component platform 380 provides connectivity by way of the bus 560 amongst some or all of the components and secures a variety of components including, for example, a pressure source 505, a control unit 510, a power source 515, a pressure sensor 520, a communications interface 525 and/or a data store 530. In the variation of FIG. 6, the component platform 380 can include first and second pressure sensors 520, 535 and first and second temperature sensors 540, 545. A reference chamber 550 can also be placed on the component platform 370 which has a known volume and in which the second pressure sensor 535 and the second temperature sensor 545 are disposed therein (or otherwise positioned as to make measurements within the reference chamber 550). The component platform 370 can also have mechanical interfaces 390 which connect to or correspond with the on/off buttons 320 and the various external settings elements 330.

The pressure source 505 is a device or collection of device for generating pressure. The pressure source 505 can, for example, be a piezoelectric pneumatic gas pump or other pneumatic pump which is positioned or includes channels (i.e., tubes) to cause air (or other gas) be expelled into the pressure chamber 160 by way of the valving mechanism 370. The pressure source 505 can operate within varying ranges such as 0.1 to 40 PSI, or between 0.1 to 300+ PSI, and is controlled through signaling from the control unit 510. The valving mechanism 370 can comprise a one-way valve which may or may not include a pressure check component. The valving mechanism 370, in some variations, comprises complementary flaps which are biased in a closed position and which open in response to gas being expelled by the pressure source 505.

The control unit 510 comprises one or more processors (e.g., CPUs, GPUs, etc.) and memory storing instruction for execution by the one or more processors. The control unit 510 can include one or more programmable controllers (e.g., microcontrollers, etc.). The control unit 510 can execute operations or otherwise generate signals for execution by other components such as the pressure source 505 and the pressure sensor 520 as well as receive signals from the various components. For example, the control unit 510 can send signals to change pressure limits from a first value to a second value or to change infusion from a first profile (a first length of time at a first pressure) to a second profile (a second, different length of time at a second, different pressure).

In some variations, the control unit 510 can estimate and correlate pressures with volume infused, flow rate, and therapy settings relative to a type of the fluid and/or an infusion protocol. The control unit 510 can selectively send signals to the pump 510 to address various requirements including: maintaining a constant flow rate, varying flow rate, open or closed loop system control, biological signals that measure patient response during a therapy session, modifications of therapy parameters based on progression of patient along a therapy regime over the course of time, and other adjustable programs based on patient, clinician, pharmacy assessment of individual patient needs and the like. The control unit 510 can be programmed locally or remotely (e.g. on a user interface of a remote computing device in communication with the control unit 510 by way of the communications interface 525).

The pressure values generated by the pressure sensor 520 can be used by the control unit 510 to determine when all of the fluid in the medication container 120 has been expelled through the orifice 126. For example, the pressure remaining constant may indicate that all of the fluid has been expelled. In addition, the measured pressure can be used by the control unit 510 to determine if there is an occlusion condition within an infusion set (e.g., tubing 140 and/or needle set 150, etc.) coupled to the medication container 120. For example, spike or other pre-defined pattern in the measured pressure level may be indicative of an occlusion. The control unit 510 can, for example, use pattern matching or other signal processing techniques to correlate measured pressure profiles with empirically measured occlusion pressure profiles. Further, the control unit 510 can also determine, based on the measured pressure from the pressure sensor 520, that the medication container 120 has been removed from the medication container interface 340. For example, the pressure in the pressure chamber 160 may be vented when the medication container 120 has been removed which can be detected by the measured level of pressure by the pressure sensor 515.

The control unit 510 can be configured to allow for user-assisted or automatic priming of the medication container 120 to remove any bubbles and the like from the medication chamber 130 and from the fluid path to the patient. With the user-assisted approach, the user can activate the pressure source 505 (optionally through activation of the one of the buttons 320/330 and/or using a remote computing device such as a mobile phone, tablet, patient monitor, etc.) to expel fluid and/or gas causing the fluid to approach the terminus of the fluid path (i.e., the point at which fluid enters the patient, etc.). The terminus of the fluid path can, for example, be a needle(s) forming part of a needle set 150 as illustrated in FIGS. 1 and 2. In other cases, the terminus of the fluid path can be a needle or other injection device directly coupled to the infusion pump 110. The user can then deactivate the pressure source 505 once the user visually confirms that the fluid within the medication chamber 130 just prior to reaching the terminus of the fluid path. With the automatic approach, the control unit 510 can be configured to control the pressure source 505 to expel gas and/or fluid until such time as the fluid within the medication chamber 130 just prior to reaching the terminus of the fluid path. With such an arrangement, the control unit 510 can utilize a look up table corresponding to the type of medication container 120 and corresponding fluid attributes with likely priming requirements. In other variations, the control unit 510 can receive signals from a bubble sensor within or adjacent to the housing 310 which can optically detect whether there the medication chamber 130 is fully or adequately primed (i.e., bubbles have been expelled, etc.). In either approach, the control unit 510 can be cause the pressure source 505 to operate at a lower level than during a typical infusion to avoid or otherwise minimize the likelihood of fluid within the medication chamber 130 crystallizing when exiting the terminus of the fluid path.

The power source 515 can be a local power source (e.g., a rechargeable or non-rechargeable battery) or it can be a connector to an external power source (e.g., a hard-wired AC/DC adapter, etc.). In some cases, the amount of remaining charge of the power source 515 can be detected and conveyed to a user. Audio/visual alerts can be generated when the remaining charge is below a certain level and/or is insufficient to complete the current infusion.

The pressure sensor 520 can be positioned near or adjacent to the pressure source 505 and can measure pressure within the pressure chamber 160. In some variations, the system works in an open loop manner (as described in further detail below) in which case values generated by the pressure sensor 520 can be used to reference look up tables that establish flow rates as correlated with pressures given other attributes including, for example, biological parameters, temperature and accessories.

The communications interface 525 can be a wireless interface which allows the infusion pump to bi-directionally communicate over one or more networks and/or in a peer-to-peer fashion (e.g., Bluetooth, etc.) to external computing devices. In some variations, the communications interface 525 is a wired connection to a network, router, etc. The external computing devices which can exchange signals/data with the infusion pump 110 can take various forms including a mobile phone, tablet, remote computer, patient monitor, hospital data system and the like which can include an interface for monitoring, logging, and controlling infusions by the infusion pump 110 (e.g., change the operational parameters associated with the settings elements 330, etc.). The communications interface 525 in some variations can communicate with other external sensors or medical devices which are used in connection with the care of the patient receiving the infusion. Example sensors or medical devices include in-line flow sensors, in-line pressure sensors, physiological sensors measuring a patient vitals, and the like. In some cases, data received by way of the communications interface 525 from the external sensors/medical devices can be used by the control unit 510 to alter or otherwise change one or more operational parameters of the pneumatic pump which, in turn, alters how fluid within the medication container 120 is being infused to the patient. In some variations, the infusion pump 110 does not necessarily require the pressure sensor 520 and can obtain signals by way of the communications interface 525 from an external pressure sensor or flow rate sensor which can be positioned, for example, downstream from the medication container 120 along the tubing set 140.

The communications interface 525 can be used to transmit data characterizing the infusion such as fluid flow rate information or values to calculate the fluid flow rate information. Fluid flow rate information can take varying forms including one or more of time of infusion (as based on a clock of one of the components 505-530), fluid flow rate, fluid pressure profile (i.e., the pressure measurements during the course of a single infusion), mode of operation by the pneumatic pump (i.e., which setting element 330 was activated, etc.), and patient compliance statistics. The ability provided by the current subject matter to monitor patient therapy adherence each session and over time is an important adjunct to clinical optimization for each patient including prevention of medicine loss or misuse. Collection of statistical data generated by or in conjunction with the infusion pump 110 including therapy compliance and response for groups of patients can provide substantive enhancements to subsequent prescriptions as well as feedback to pharmacies about patient management and feedback to pharmaceutical companies about patient response to their respective drugs. In some cases, the communications interface 525 can receive signals external to the infusion pump 110 which would cause the control unit 510 to, in turn, cause the pressure source 505 to cease (thus ending an infusion).

The data store 530 can log information generated by the pressure source 505 and/or the pressure sensor 520 and the like in order to characterize infusion parameters. This logged information can be transmitted to a remote computing device by way of the communications interface 525 to, for example, track compliance with prescribed regimens and the like.

In some variations, the housing 310 can include an air port 395 which can be used to supply gas to a tubing set or other device (e.g., vial medication transfer device, wearable injectors, injector pen, etc.) coupled directly or indirectly to the air port 395. The air port 395 can, in some variations, include a Luer lock to connect to such a tubing set or other device. Other connecting mechanisms can be provided depending on the downstream use of the air port. In some variations, the air port 395 is coupled to a tubing set extending therefrom. The gas supplied through the air port 395 can be by way of a channel coupled to the pump 510. This channel can be dedicated or selectively activated by way of valving within the housing. In other cases, the gas supplied through the air port can be generated by a second pump.

In some variations, a proximity sensor in or adjacent to the medication container interface 340 can be used which is configured to detect presence or absence of a medication container 120 within the medication container interface 340 and transmit corresponding signals to the control unit 510. The proximity sensor, in some implementations, can be an additional sensor 555 forming part of the component platform 380.

In some variations, a position sensor in or adjacent to the medication container interface 340 can be used which is configured to detect changes correlated to a volume of the medication chamber 130 (e.g., position of the stopper 124, etc.) and transmit corresponding signals to the control unit. The position sensor, in some implementations, can be an additional sensor 555 forming part of the component platform 380. The position sensor, in some variations, can visually determine and characterize movement of the plunger 124 (or other elements of the medication container 120 indicative of flow rate or remaining fluid, etc.) which can be used to calculate change in volume over time. The position sensor can be or include various sensors such as a linear displacement encoder, time-of-flight laser or ultrasound, or digital camera.

In some variations, the pressure sensor 520 can be omitted and a lookup table can be used (and optionally stored in the data store 530) which maps flow rate to viscosity of the fluid being administered.

The current subject matter is advantageous in that the infusion pump 110 can use medication containers 120 having varying sizes (and thus different barrel diameters). The force applied to a plunger 124 creates fluid pressure inside the medication container 120, which drives the fluid through orifice 126 to the tubing set 140/needle(s) 150. The resulting flow rate is directly influenced by this pressure. If the applied force remains constant but the barrel diameter changes, the fluid pressure varies inversely with the cross-sectional area of the barrel. Specifically, a larger barrel diameter reduces the pressure (and thus the flow rate), while a smaller diameter increases the pressure (and flow rate), due to the relationship P=F/A, where P is pressure, F is force, and A is the area of the syringe barrel.

By having the pressure source 505 generate constant air pressure as the driving mechanism (instead of a fixed mechanical force) different barrel sizes can be used as the air pressure applied to the fluid by way of the plunger 124 remains uniform regardless of the barrel diameter. This arrangement ensures that the fluid pressure, and consequently the flow rate, stays consistent even if the medication container barrel diameter varies, providing more reliable and predictable drug delivery.

The infusion pump 110 can have an electronic, closed-loop firmware control of the applied air pressure (by way of the pressure source 505) that can be adjusted via a user interface on a remote computing device or on setting elements 330. Air pressure applied to the plunger 124 (e.g., syringe seal, etc.) can be measured using the pressure sensor 520. The user-selected variable pressure setting (e.g., settings 1-5, etc.) allows a range of flow rates to be achieved without the need for flow control/restrictor devices along the tubing set 140. In some use cases, the applied pressure generated by the pressure source 505 can be fixed (not allowing the user to make adjustments). In some cases, the control unit 510 can cause the pressure generated by the pressure source 505 to be ramped, staged, or have different profiles depending on the infusion therapy requirements (e.g., ramped infusion rate or boluses). In particular, the setting elements 330 can provide user-selected variable pressure setting which allow a range of flow rates to be achieved without the need for flow control/restrictor devices. In some use cases, the applied pressure can be fixed (not allowing user to adjust). In some cases, the pressure can be ramped, staged, or have different profiles depending on the infusion therapy requirements (e.g., ramped infusion rate or boluses).

The use of near-constant applied air pressure (i.e., intermittent on/off of the pressure source 505 to maintain a set target pressure within a certain threshold/tolerance) with the use of feedback from the pressure sensor 520 allows for variable pressure control, volume estimation, flow rate control, syringe insertion/presence or removal detection, and end of infusion/occlusion detection, depending on the implementation and use application. The pressure source 505 duty cycle also helps to extend the device run time on a single charge of the internal battery (i.e., power source 515).

The control unit 310 can be used to control flow rate in either an open-loop or a closed-loop fashion, depending on the particular configuration and use application. Flow rate can be controlled by the control unit 310 modulating the applied pressure/force on the plunger 124 to achieve the desired flow rate. Flow rate can be measured or estimated by various techniques. In one variation, the flow rate can be determined based on the pressure applied and the viscosity of the medicine through a pre-recorded table (530). In another variation, flow rate can be determined using the ideal gas law equation (PV=nRT, P₁V₁/T₁=P₂V₂/T₂) with pressure measurements and a known reference volume. In particular, a first pressure sensor 520 can measure pressure within the pressure chamber 160 and generate a first pressure value while a second pressure sensor 535 can measure pressure within a reference chamber 550 encapsulated within the housing 310 and generate a second pressure value. The control unit 310 can calculate an amount of fluid expelled from the medication container 120 based on a differential pressure measurement using the first pressure values and the second pressure value and leveraging the ideal gas law equation. In some cases, the temperature can be assumed to be the same. In other variations, a first temperature sensor 540 can measure temperature within the pressure chamber 160 and a second temperature sensor 545 can measure temperature within the reference chamber 550. Thus, the respective pressure and temperature values within the pressure chamber 160 and the reference chamber 550 can be used to calculate the volume of the pressure chamber 160 (which can then be correlated to amount of fluid expelled from the medication chamber 130).

In some variations, the housing 310 can encapsulate a pressure relief valve which is in communication with the pressure chamber 160. This pressure relief valve can, for example, be mechanical in that it releases pressure when a certain physical parameter condition (e.g., pressure value, etc.) is met. Alternatively, the pressure relieve valve can be electromechanical (e.g., a solenoid valve) which can be opened in response to a signal from the control unit 510. The pressure relief valve, in some variations, forms part of the component platform 380.

As noted above, while the current description is largely directed to the use of a plunger-less syringe, different types of medication containers 120 can be used. For example, the infusion pump 110 can be used in connection with an on-body injector which is adjustable to a contour of a body of a user. Such an on-body injector can have a dual chamber arrangement; namely a pressure chamber in combination with a medication chamber. With this arrangement, the air port 395 can be used to supply pressure to the pressure chamber. As another example, the medication container 120 can be an injector pen in which. The injector pen would also have a dual chamber in which the air port 395 is used to supply pressure to the corresponding. As yet another example, the medication container 120 can be a flat reservoir or bag in which pressure is applied directly or indirectly to a deformable/moveable surface of such reservoir or bag. In some variations, there is a dual chambers approach (pressure chamber in combination with medication chamber) with different form factors to elongate (pen) or flatten (onbody) as needed. Still further, the medication container 120 can be a vial in which an opening of the vial can be secured to the medication container interface 340 or alternatively an intermediate adapter (e.g., vial adapter transfer device) can be coupled to the opening of the vial and secured to the medication container interface 340. The vial adapter transfer device can, for example, by a spike that allows the infusion pump 110 to pump gas into the vial while allow the fluid contained therein to be pushed through a second channel (which can be part of the same spike) out of the vial into the tubing 140/needle set 150.

With the plunger-less syringe variation of the medication container 120 described above, the pressure generated by the pressure source 505 is directedly applied to the surface of the medication container 120 (i.e., the stopper 124). In other variations, pressure generated by the pressure source 505 is indirectly applied to the surface of the medication container 120 by way of one or more intermediate elements between the housing 310 and the medication container 120. For example, the pressure source 505 can inflate an expandable element (e.g., balloon, etc.) within the pressure chamber 160 which causes the fluid to be expelled out of the orifice 126 of the medication container 120. In the variation of a plunger-less syringe, the expandable element can contact the stopper 124.

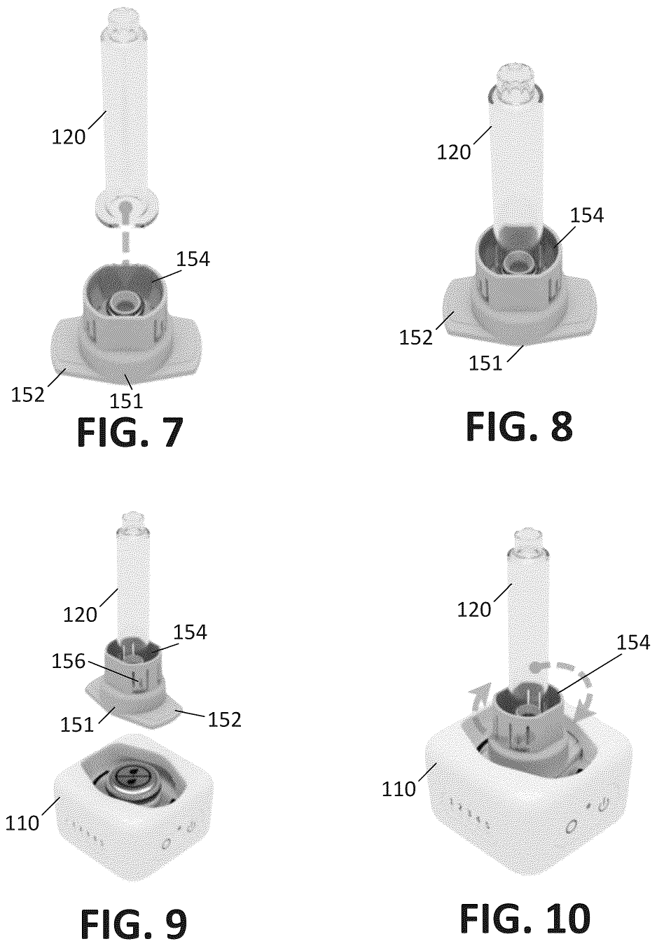

In some variations, the medication container 120 may have a shape and size that is not directly compatible with the medication container interface 310. In such cases and with reference to FIGS. 7-10, an adapter 150 can be utilized. The adapter can include flanges 152 extending therefrom to secure with the medication container interface. The adapter 150 can also include as a receiving cavity 154 into which the medication container is inserted. The housing of the adapter 150 can also include securing elements 156 (e.g., locking tabs, snaps, etc.) which can secure the medication container 120 after it is placed therein. The medication container 120 in FIGS. 7-10 omits the stopper 124 for ease of illustrated. FIG. 7 illustrates the medication container 120 and adapter 150 prior to coupling. FIG. 8 illustrates the medication container 120 after it is inserted into and secured by the adapter 150. FIG. 9 illustrates the combination of the medication container 120 and adapter 150 relative to the infusion pump. FIG. 10 illustrates the process of securing the medication container 120/adapter 150 combination into the infusion pump. Like the earlier examples, the medication container 120 and adapter 150 form a pressure chamber 160 relative to the mount 350 and seal 360.

Various implementations and aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor and memory, which may be special or general purpose, coupled to receive data and instructions from, and to transmit data and instructions to, a storage system, at least one input device, and at least one output device.

Although a few variations have been described in detail above, other modifications are possible. For example, the logic flow depicted in the accompanying figures and described herein do not require the particular order shown, or sequential order, to achieve desirable results. Other embodiments may be within the scope of the following claims.

1. An apparatus for delivering medication comprising:
a housing defining a medication container interface having a shape and size to interface with a first portion of a medication container, the medication container comprising a second portion having an orifice and a medication chamber, the medication chamber encapsulating fluid and is disposed between the first and second portions of the medication container, a pressure chamber being formed when the first portion of the medication container is interfaced with the medication container interface;
a pressure source encapsulated within the housing which, when active, expels gas and/or fluid within the pressure chamber to apply a pressure to a surface of the medication container causing fluid within the medication chamber to be expelled out of the orifice and administered to a patient by way of an infusion set;
a pressure sensor within the housing to measure pressure within the pressure chamber; and
a control unit in communication with the pressure source and the pressure sensor which, based on the measured pressure, sends signals to the pressure source to control infusion parameters;
wherein the medication container interface comprises a mount having stacked, stepped ridges each with a corresponding seal disposed on an outer surface of the mount, the seals being configured to create an air-tight seal with an opening of the first portion of the medication container to form the pressure chamber.

2. The apparatus of claim 1, wherein the first portion of the medication container comprises a movable surface or component.

3. The apparatus of claim 1, wherein the first portion of the medication container comprises a compressible or deformable surface.

4. The apparatus of claim 1, wherein the pressure source is a pneumatic pump.

5. The apparatus of claim 4, wherein the pneumatic pump comprises a piezoelectric pump.

6. The apparatus of claim 1, wherein the medication container is directly coupled to the medication container interface.

7. The apparatus of claim 1, wherein the medication container is indirectly coupled to the medication container interface.

8. The apparatus of claim 1, wherein the pressure is directly applied to the surface of the medication container.

9. The apparatus of claim 1, wherein the pressure is indirectly applied to the surface of the medication container by way of one or more intermediate elements between the housing and the medication container.

10. The apparatus of claim 1, wherein at least a portion of the medication container interface is integral to the housing.

11. The apparatus of claim 1, wherein the medication container interface is an external component secured to the housing.

12. The apparatus of claim 1, wherein the medication container is a syringe comprising at least one flange on the first portion and the medication container interface comprises one or more locking tabs positioned to allow insertion and securing of the first portion of the medication container into the medication container interface, the medication container being secured by the one or more locking tabs after rotating from a first position to a second position.

13. The apparatus of claim 1 further comprising:
the medication container, the medication container having a flange on its first side; and
an adapter having one or more locking tabs positioned to allow insertion and securing of the first side of the medication container into the adapter, the adapter having a shape and size to be received in and secured by the medication container interface.

14. The apparatus of claim 1, wherein the control unit comprises a programmable controller.

15. The apparatus of claim 14, wherein the control unit controls infusion parameters using a look up table that correlates one or more of detected pressure, a geometry of the medication container, a viscosity of fluid contained within the medication container, and attributes of an infusion tubing set in communication with the orifice with a predetermined flow rate.

16. The apparatus of claim 15 further comprising:
a data store encapsulated within the housing to store infusion parameter measurements generated by the control unit;
a central processing unit (CPU);
memory within the housing to store infusion parameters generated by the control unit and the pressure sensor and time measurements generated by the CPU; and
a communications interface encapsulated within the housing and in communication with the data store and the control unit, the communications interface transmitting data stored in the memory characterizing the infusion parameter measurements to a remote computing device.

17. The apparatus of claim 16, wherein a remote computing device is configured to send signals, by way of the communications interface, to initiate and stop expulsion of the fluid from the medication container.

18. The apparatus of claim 17, wherein the remote computing device is a mobile phone, a tablet, a patient monitor, a computer, or a hospital data system.

19. The apparatus of claim 16, wherein the transmitted data comprises fluid flow rate information or values to calculate the fluid flow rate information.

20. The apparatus of claim 19, wherein the fluid flow rate information comprises one or more of: time of infusion, fluid flow rate, fluid pressure profile, mode of operation by the pressure source, and patient compliance statistics.

21. The apparatus of claim 1, wherein the control unit sends a first signal to the pressure source to cause the fluid to be expelled out of the orifice according to a first flow rate profile and subsequently sends a second signal to the pressure source to change a fluid pressure profile at which the fluid is expelled out of the orifice from the first flow rate profile to a second, different flow rate profile.

22. The apparatus of claim 1, wherein the control unit, based on the measured pressure, and fluid characterization data, determine that all of the fluid in the medication container has been expelled through the orifice.

23. The apparatus of claim 1, wherein the control unit, based on the measured pressure, determines an occlusion condition within an infusion set coupled to the medication container based on a detected build up of pressure within the pressure chamber.

24. The apparatus of claim 1, wherein the control unit, based on the measured pressure, determines that the medication container has been removed from the medication container interface based on a detected release of pressure within the pressure chamber.

25. The apparatus of claim 1 further comprising:
a reference chamber having a known volume;
a first temperature sensor within the housing and in communication with the control unit to measure temperature within the pressure chamber; and
a second temperature sensor within the housing and in communication with the control unit to measure temperature within the reference chamber;
wherein the infusion parameters are further controlled based on values generated by the first temperature sensor and the second temperature sensor.

26. An apparatus for delivering medication comprising:
a housing defining a medication container interface having a shape and size to receive and secure a first portion of a medication container, the medication container further comprising an orifice on a second portion of the medication container, a pressure chamber being formed when the first portion of the medication container is secured to the medication container interface;
a pressure source encapsulated within the housing which, when active, expels gas and/or fluid within the pressure chamber to apply a pressure to a surface of the medication container causing fluid within the medication container to be expelled out of the orifice and administered to a patient by way of an infusion set;
a pressure sensor configured to measure the pressure within the pressure chamber; and
a control unit comprising a programmable microprocessor in communication with the pressure source and the pressure sensor, the control unit being configured to:
receive a first pressure value at a first time and a second pressure value at a second time separated by a sampling interval from the pressure sensor;
determine, using a stored relationship between pressure in the pressure chamber and volume of gas in the pressure chamber, a change in chamber volume corresponding to an expelled volume from the medication container;
compute a flow rate based on the expelled volume and the sampling interval;
compare the computed flow rate to a target flow rate; and
adjust an operating parameter of the pressure source to reduce a difference between the computed flow rate and the target flow rate so as to maintain a constant flow rate.

27. The apparatus of claim 26 further comprising: at least one pressure sensor to measure pressure within the pressure chamber.

28. An apparatus for delivering medication comprising:
a housing defining a medication container interface having a shape and size to receive and secure a first portion of a medication container, the medication container further comprising an orifice on a second portion of the medication container, a pressure chamber being formed when the first portion of the medication container is secured to the medication container interface;
a pressure source encapsulated within the housing which, when active, expels gas and/or fluid within the pressure chamber to apply a pressure to a surface of the medication container causing fluid within the medication container to be expelled out of the orifice and administered to a patient by way of an infusion set;
a reference chamber having a known volume;
a first pressure sensor within the housing to measure pressure within the pressure chamber and generate a first pressure value;
a second pressure sensor within the housing to measure pressure within the reference chamber and generate a second pressure value;
a first temperature sensor within the housing and in communication with the control unit to measure temperature within the pressure chamber and generate a first temperature;
a second temperature sensor within the housing and in communication with the control unit to measure temperature within the reference chamber and generate a second temperature; and
a control unit in communication with the pressure source and the first pressure sensor, the second pressure sensor, the first temperature sensor, and the second temperature sensor which, based on the first pressure value, the second pressure value, the first temperature, and the second temperature, sends signals to the pressure source to control infusion parameters so as to maintain a constant flow rate.

29. (canceled)

30. The apparatus of claim 28, wherein the control unit determines an occlusion or infusion completion state based on a level of measured pressure in the pressure chamber.

31. The apparatus of claim 28, wherein the control unit is configured to execute an automatic priming protocol comprising:
initiating priming in response to a user activation or an automatically initiated priming mode;
actuating the pressure source to expel gas and/or fluid through the medication container and a fluid path toward a terminus of the fluid path;
operating the pressure source at a level lower than during a typical infusion to minimize crystallization at the terminus;
monitoring, during priming, pressure measured in the pressure chamber by the first pressure sensor; and
ceasing actuation of the pressure source when the control unit determines, based on at least one of (i) a lookup table that correlates measured pressure, a type of the medication container, fluid attributes, and infusion set attributes with priming requirements, and (ii) a signal from a bubble sensor indicating adequate priming, that fluid within the medication chamber is just prior to reaching the terminus of the fluid path.
